(19) United States
(12) Patent Application Publication
(10) Pub. No.: US 2003/0084477 A1
(43) Pub. Date: May 1, 2003

(54) Genes enhancing disease resistance in plants

(76) Inventors: Gregory B. Martin, Ithaca, NY (US); Jian-Min Zhou, Manhattan, KS (US)

Correspondence Address: Woodard, Emhardt, Naughton, Moriarty and McNett, Bank One Center/Tower, Suite 3700, 111 Monument Circle, Indianapolis, IN, 46204-5137, US

(21) Appl. No.: 10/122,822

(22) Filed: Apr. 15, 2002

Related U.S. Application Data
(63) Continuation of application No. 09/202,161, filed on Jun. 14, 1999.

(30) Foreign Application Priority Data
Jun. 12, 1997 PCT/US97/10382

(51) Int. Cl. A01H 5/00; C12N 1/21; C12N 9/50

(52) U.S. Cl. 800/279; 800/294; 435/219; 435/320.1; 435/252.2

(57) ABSTRACT

The present invention relates to methods and materials for the protection of plants against pathogens through plant genetic engineering; and more particularly to genes which enhance disease resistance in plants by encoding proteins that physically interact with R gene products involved in activation of plant defense mechanisms. The invention further relates to three nucleotide sequences which have been cloned, isolated and sequenced, three amino acid sequences encoded thereby and a transgenic plant and methods for making the same, the genome of the plant having incorporated therein a foreign nucleotide sequence selected in accordance with the invention which functions to enhance the plant's ability to resist pathogens.

### REFERENCES TO RELATED APPLICATIONS

This application claims the benefit of U.S. Provisional Application No. 60/091,633, filed Jun. 12, 1996, and U.S. Provisional Application entitled THE PTO KINASE CONFERRING RESISTANCE TO TOMATO BACTERIAL SPECK DISEASE INTERACTS WITH PROTEINS THAT BIND A CIS-ELEMENT OF PATHOGENESIS-RELATED GENES, filed May 14, 1997, each of which is hereby incorporated by reference herein in its entirety.

This invention was made with government support under the following grant: grant number MCB-96-30635 awarded by NSF. The government has certain rights in the invention.

### BACKGROUND OF THE INVENTION

1. Field of the Invention

The present invention relates to methods and materials for the protection of plants against pathogens through plant genetic engineering. More particularly, the invention relates to genes which enhance a plant's ability to withstand pathogen attack by encoding proteins that physically interact with proteins encoded by disease resistance genes (R genes) in a plant's signal transduction pathway to activate plant defense mechanisms. The invention also relates to transgenic plants and methods for making the same, the genomes of the plants having incorporated therein foreign nucleotide sequences selected in accordance with the invention which function to enhance the plants ability to resist pathogens.

2. Discussion of Related Art

Crop losses resulting from pathogenic organisms such as viruses, bacteria, fungi and nematodes is a historic and widespread problem in a wide variety of agricultural industries. These crop losses caused by pathogen-related plant damage result in economic losses amounting to billions of dollars annually. This problem has been addressed in the past by employing a wide variety of chemicals to reduce pest damage to plant crops. The approach, however, has been associated with many environmental problems created by the widespread use of pesticidal chemicals, and the chemicals often only provide a transient level of protection for crops. Chemicals also suffer from the disadvantage that all organisms in an area may be indiscriminately treated, causing needless damage to many beneficial organisms. Perhaps more importantly, many chemicals are potentially toxic to man and animals and often become concentrated in, for example, lakes and ponds and/or other water supplies.

As a result, alternate methods have been explored to reduce crop damage, one example being selective breeding of plants based upon pathogen resistance characteristics. Resistance traits, however, are sometimes controlled by many genes, making it difficult to genetically select a desired attribute to a satsfactory degree. Decreased crop yields are also occasionally encountered in resistant plants developed by selective breeding. Accordingly, there exists a strong need for compositions and methods to improve the resistance of plants from attack by pathogens. Such are provided by the present invention, which provides compositions and methods useful for genetically transforming a plant and thereby enhancing the plant's resistance to pathogen attack.

A transgene, such as a nucleotide sequence selected in accordance with the present invention, is expressed in a transformed plant to produce in the cell a protein encoded thereby. Briefly, transcription of the DNA sequence is initiated by the binding of RNA polymerase to the DNA sequence s promoter region. During transcription, movement of the RNA polymerase along the DNA sequence forms messenger RNA (“mRNA”) and, as a result, the DNA sequence is transcribed into a corresponding mRNA. This mRNA then moves to the ribosomes of the rough endoplasmic reticulum which, with transfer RNA (“tRNA”), translates the mRNA into the protein encoded thereby. Proteins of the present invention thus produced in a transformed host then perform an important function in the plant's signal transduction pathway corresponding to pathogen resistance. Although the sequence of events involved in the resistance mechanism is not well understood, it is clear that proteins contemplated by the present invention enhance a plant's resistance response by participating in this signal transduction pathway.

To comment generally upon plant resistance to pathogens, plants respond to pathogen infection in various ways, including a rapid induction of localized necrosis at the site of infection (the hypersensitive response, HR), production of antimicrobial compounds, lignin formation, oxidative burst, and increased expression of defense-related genes. Two categories of genes and, therefore, proteins are involved in a plant's response system, disease resistance (R) genes and defense genes. R genes typically encode proteins which play a role in pathogen recognition and/or signal transduction.

R genes may be identified based upon their polymorphism in a particular plant species. That is, some crop varieties contain a particular R gene and others will lack that gene. Analysis of the progeny of genetic crosses between resistant and susceptible crop varieties allow the mapping of R genes to specific regions on a chromosome. R genes frequently, although not always, display dominant gene action and play a major qualitative role in conferring disease resistance. They frequently map to single loci in the genome and are often found to be members of a gene family. R genes differ from other genes that may play a role in disease resistance later in the defense response (after pathogen recognition). These other “downstream” genes are often referred to as “defense genes” or “defense-related genes” and include the class of genes known as “pathogenesis-related” (PR) genes.

With regard to increased expression of defense-related genes, it has long been recognized that transcriptional activation of a battery of plant defense-related genes is commonly associated with pathogen invasion. Defense genes include, for example, those encoding pathogenesis related proteins (PRs), hydroxyproline rich glycoproteins and enzymes for phytoalexin biosynthesis such as phenylalanine ammonia lyase (PAL) and chalcone sythase. Although the role of these proteins in plant disease resistance is not well understood, their enzymatic functions indicate that they are well suited for defense against pathogens. Results of preliminary research have spurred extensive investigations into the biological function of defense genes and mechanisms by which they are activated.

With respect to R genes, it has been postulated that disease resistance of a plant may be induced by the genetic interaction of single genes in both the pathogen and the plant host. The phenomenon of disease resistance is believed to be initiated by physical contact between a pathogen and a potentially compatible portion of the host. Once such contact has occurred, usually as a result of wind or rain vectored deposition of the pathogen, the pathogen must recognize that such contact has been established in order to initiate the pathogenic process. Likewise, such recognition by the host is required in order to initiate a resistance response. A great deal of research is currently focused upon elucidating the precise manner in which such recognition occurs. Pathogen recognition is believed to be associated with low pH of plant tissues or the presence of plant-specific metabolites. It is believed that plant recognition occurs as a result of a race-specific mechanism where the protein product of a host disease resistance (R) gene recognizes the product of an avirulence gene of the pathogen. As a result, the plant's defense responses are activated, leading to production of various factors (e.g., gum or cork production, production of inhibitors of pathogen proteases, deposition of lignin and hydroxyproplin-rich proteins in cell wails) and offensive resistance factors (e.g., production of phytoalexins, secreted chitinases). If the rate and level of activation of the genes producing these factors is sufficiently high, the host is able to gain an advantage on the pathogen. On the other hand, if the pathogen is fully activated at an earlier stage in the infection process, it may overwhelm both the offensive and defensive resistance factors of the plant.

In this regard, much effort has been focused on the characterization of cis-acting elements involved in elicitor- and pathogen-induced defense gene expression, and a few putative transcription factors involved in defense responses have been identified. Many defense-related genes are induced in both compatible (susceptible) and incompatible (resistant) plant-pathogen interactions. However, the expression of many defense genes is more rapid and pronounced in a plant challenged with an incompatible pathogen. In many plant-pathogen interactions, these defense responses are activated upon recognition of a pathogen carrying a specific avirulence (avr) gene by a plant host containing a corresponding R gene. In particular, incompatible interactions involving a plant R gene and a corresponding pathogen avr gene lead to accelerated plant defense gene expression. Many R genes encode proteins that are likely involved either in the recognition of signals determined by avr genes or in the early steps of signal transduction. However, a direct link between any R gene and defense gene activation has not previously been established.

In tomato, resistance to the bacterial pathogen Pseudomonas syringae pv. tomato (which causes bacterial speck disease) has been shown to be associated with a single locus (Pto) that displays dominant gene action. Resistance of plants carrying the Pto locus to Pseudomonas syringae pv. tomato strains expressing the avirulence gene avrPto is a model system for signal transduction pathways mediated by a specific R gene. This system constitutes the only example of R gene mediated resistance pathway in which genes for multiple components have been cloned. Currently, three componenets are known to be involved in the signaling pathway mediated by Pto: the serine/threonine protein kinase Pto, a second serine/threonine kinase Pti1, and the leucine-rich-repeat type protein Prf. The Pto gene was originally discovered in Lycopersicon pimpinellifolium, a wild tomato species, and isolated by map-based cloning. Mutagenesis of a bacterial speck-resistant tomato line revealed a second gene, Pr/f that is required for both Pto-mediated resistance and fenthion sensitivity, a related phenotype mediated by the Fen gene. Using the yeast two-hybrid system with Pto as a bait, the present inventors have identified another protein kinase Pti1 that appears to act downstream of Pto and is involved in the hypersensitive response.

In accordance with the present invention, three additional Pto-interacting proteins, Pti4, Pti5 and Pti6, also referred to herein as Pti4/5/6, that belong to a large family of plant transcription factors, are characterized. These proteins bind to a cis-element that is widely conserved among “pathogenesis-related” (PR) genes and are implicated in the regulation of these genes during incompatible plant-pathogen interactions. Pti4/5/6 each have characteristics that are typical of transcription factors. The present inventors have discovered that Pti4/5/6 specifically recognize and bind to a DNA sequence that is present in the promoter region of a large number of genes encoding PR proteins. Therefore, a direct connection has been discovered between a disease resistance gene and the specific activation of plant defense genes.

### SUMMARY OF THE INVENTION

The present invention relates to the isolation, purification and use of nucleotide sequences, such as, for example, Pti4, Pti5 and Pti6 (“Pti4/5/6”), which are useful for enhancing a plant's ability to resist pathogen-related disease by encoding transcription factors that enhance a plant's ability to activate defense mechanisms when faced with pathogen activity. Proteins encoded by Pti4/5/6 are useful for enhancing a plant's ability to resist pathogen attack. The proteins encoded by the Pti4/5/6, nucleotide sequences each possess a DNA binding domain, putative nuclear localization sequences (NLS) and regions rich in acidic amino acids.

It is presently shown that the newly-isolated DNA sequences of Pti4/5/6 encode transcription factors which physically interact with Pto kinase. The present invention provides a novel form of plant protection against many types of pathogens including viruses, bacteria and fungi. While it is not intended that the present invention be limited by any mechanism whereby it achieves its advantageous result, it is believed that manipulation of these transcription factors enables the coordinate regulation of large numbers of genes involved in plant disease resistance. The invention therefore, features the DNA sequences of the Pti4/5/6 genes and the amino acid sequences of the Pti4/5/6 proteins, as set forth herein, as well as DNA sequences and amino acid sequences having substantial identity thereto and having similar levels of activity. Inventive genes may be inserted into an expression vector to produce a recombinant DNA expression system which is also an aspect of the invention.

In one aspect of the invention, inventive DNA sequences conferring disease resistance to plants are used to transform cells and to transform plants. In another aspect of the invention, there is provided a process of conferring disease resistance to plants by growing plant cells transformed with an inventive recombinant DNA expression vector and capable of expressing the DNA sequences. Plants transformed with inventive nucleotide sequences thereby have an enhanced ability to resist attack by pathogens which have an avr gene corresponding to a plant resistance gene.

It is an object of the present invention to provide isolated, sequenced and purified proteins which are useful for conferring disease resistance to a plant.

Another object of the invention is to provide isolated nucleotide sequences which encode said proteins and thereby find advantageous use when incorporated into a vector or plasmid as a transformant for a plant or microorganism.

Additionally, it is an object of the invention to provide transformed plants which have enhanced ability to resist attack by pathogens.

Further objects, advantages and features of the present invention will be apparent from the detailed description herein.

### BRIEF DESCRIPTION OF THE FIGURES

Although the characteristic features of this invention will be particularly pointed out in the claims, the invention itself, and the manner in which it may be made and used, may be better understood by referring to the following description taken in connection with the accompanying figures forming a part hereof.

FIG. 1 sets forth a comparative alignment of Pti4/5/6 amino acid sequences. The Pretty Box program (GCG package, version 7.0) was used to create the best alignment. Also set forth in FIG. 1 are amino acid consensus 1 motif (“A”) and amino acid consensus 2 motif (“B”).

FIG. 2 sets forth results of the Experiment described in Example 1 herein. Briefly, EGY48 yeast cells containing a prey of Pti4, Pti5 or Pti6 (in pJG4-5), and a bait of Pto, pto or Bicoid (in pEG202) were grown on galactose Ura⁻ His⁻ Trp⁻ X-Gal medium. The plates were incubated at 30° C. for three days and photographed. Four independent, representative colonies are shown for each bait/prey combination.

FIG. 3 sets forth the results of the gel blot analysis procedure described in Example 2 herein.

FIG. 4 sets forth the results of the gel mobility-shift assay described in Example 4 herein.

### DETAILED DESCRIPTION OF THE INVENTION

For purposes of promoting an understanding of the principles of the invention, reference will now be made to particular embodiments of the invention and specific language will be used to describe the same. It will nevertheless be understood that no limitation of the scope of the invention is thereby intended, such alterations and further modifications in the invention, and such further applications of the principles of the invention as described herein being contemplated as would normally occur to one skilled in the art to which the invention pertains.

The present invention relates to nucleotide sequences which confer disease to resistance to plants by encoding proteins that physically interact with proteins encoded by R genes to enhance the activation of plant defense genes such as, for example, PR genes. The present inventors have isolated, sequenced and characterized three biologically and commercially useful proteins (Pto-interacting proteins, or “Pti” proteins), Pti4/5/6, and have isolated, sequenced and cloned three novel nucleotide sequences which encode them. Pti4/5/6. When heightened expression of inventive nucleotide sequences is achieved in a plant in accordance with the present invention, the plant will have the improved ability to resist pathogen attack. As such, advantageous features of the present invention include the transformation of a wide variety of plants of various agriculturally and/or commercially valuable plant species to provide advantageous resistance to pathogen attack. Three amino acid sequences according to the invention are set forth in SEQ ID NO:1 (Pti4), SEQ ID NO:2 (Pti5) and SEQ ID NO:3 (Pti6) below.

| Met Asp Gln Gln Leu Pro Pro Thr Asn Phe Pro Val Asp Phe Pro Val | SEQ ID NO:1 |
|---|---|
| 1 5 10 15 | |
| Tyr Arg Arg Asn Ser Ser Phe Ser Arg Leu Ile Pro Cys Leu Thr Glu | |
| 20 25 30 | |
| Lys Trp Gly Asp Leu Pro Leu Lys Val Asp Asp Ser Glu Asp Met Val | |
| 35 40 45 | |
| Ile Tyr Gly Leu Leu Lys Asp Ala Leu Ser Val Gly Trp Ser Pro Phe | |
| 50 55 60 | |
| Asn Phe Thr Ala Gly Glu Val Lys Ser Glu Pro Arg Glu Glu Ile Glu | |
| 65 70 75 80 | |
| Ser Ser Pro Glu Phe Ser Pro Ser Pro Ala Gly Thr Thr Ala Ala Pro | |
| 85 90 95 | |
| Ala Ala Glu Thr Pro Lys Arg Arg His Tyr Arg Gly Val Arg Gln Arg | |
| 100 105 110 | |
| Pro Trp Gly Lys Phe Ala Ala Glu Ile Arg Asp Pro Ala Lys Asn Gly | |
| 115 120 125 | |
| Ala Arg Val Trp Leu Gly Thr Tyr Glu Thr Ala Glu Glu Ala Ala Ile | |
| 130 135 140 | |
| Ala Tyr Asp Lys Ala Ala Tyr Arg Met Arg Gly Ser Lys Ala His Leu | |
| 145 150 155 160 | |
| Asn Phe Pro His Arg Ile Gly Leu Asn Glu Pro Glu Pro Phe Glu Leu | |
| 165 170 175 | |
| Arg Arg Lys Gly Arg Ala Ile Gln Gly Pro Ala Ser Ser Ser Gly Asn | |
| 180 185 190 | |
| Gly Ser Met Lys Arg Arg Arg Lys Ala Val Gln Lys Cys Asp Gly Glu | |
| 195 200 205 | |
| Met Ala Ser Arg Ser Ser Val Met Gln Val Gly Cys Gln Ile Glu Gln | |
| 210 215 220 | |
| Leu Thr Gly Val His Gln Leu | |
| 225 230 | |
| Leu Val Pro Thr Pro Gln Ser Asp Leu Pro Leu Asn Glu Asn Asp Ser | SEQ ID NO:2 |
| 5 10 15 | |
| Gln Glu Met Val Leu Tyr Glu Val Leu Asn Glu Ala Asn Ala Leu Asn | |
| 20 25 30 | |
| Ile Pro Tyr Leu Pro Gln Arg Asn Gln Leu Leu Pro Arg Asn Asn Ile | |
| 35 40 45 | |
| Leu Arg Pro Leu Gln Cys Ile Gly Lys Lys Tyr Arg Gly Val Arg Arg | |
| 50 55 60 | |
| Arg Pro Trp Gly Lys Tyr Ala Ala Glu Ile Arg Asp Ser Ala Arg His | |
| 65 70 75 80 | |
| Gly Ala Arg Val Trp Leu Gly Thr Phe Glu Thr Ala Glu Glu Ala Ala | |
| 85 90 95 | |
| Leu Ala Tyr Asp Arg Ala Ala Phe Arg Met Arg Gly Ala Lys Ala Leu | |
| 100 105 110 | |
| Leu Asn Phe Pro Ser Glu Ile Val Asn Ala Ser Val Ser Val Asp Lys | |
| 115 120 125 | |
| Leu Ser Leu Cys Ser Asn Ser Tyr Thr Thr Asn Asn Asn Ser Asp Ser | |
| 130 135 140 | |
| Ser Leu Asn Glu Val Ser Ser Gly Thr Asn Asp Val Phe Glu Ser Arg | |
| 145 150 155 160 | |
| Cys | |
| Met Thr Glu Asn Ser Val Pro Val Ile Lys Phe Thr Gln His Ile Val | SEQ ID NO:3 |
| 5 10 15 | |
| Thr Thr Asn Lys His Val Phe Ser Glu His Asn Glu Lys Ser Asn Ser | |
| 20 25 30 | |
| Glu Leu Gln Arg Val Val Arg Ile Ile Leu Thr Asp Ala Asp Ala Thr | |
| 35 40 45 | |
| Asp Ser Ser Asp Asp Glu Gly Arg Asn Thr Val Arg Arg Val Lys Arg | |
| 50 55 60 | |
| His Val Thr Glu Ile Asn Leu Met Pro Ser Thr Lys Ser Ile Gly Asp | |
| 65 70 75 80 | |
| Arg Lys Arg Arg Ser Val Ser Pro Asp Ser Asp Val Thr Arg Arg Lys | |
| 85 90 95 | |
| Lys Phe Arg Gly Val Arg Gln Arg Pro Trp Gly Arg Trp Ala Ala Glu | |
| 100 105 110 | |
| Ile Arg Asp Pro Thr Arg Gly Lys Arg Val Trp Leu Gly Thr Tyr Asp | |
| 115 120 125 | |
| Thr Pro Glu Glu Ala Ala Val Val Tyr Asp Lys Ala Ala Val Lys Leu | |
| 130 135 140 | |
| Lys Gly Pro Asp Ala Val Thr Asn Phe Pro Val Ser Thr Thr Ala Glu | |
| 145 150 155 160 | |
| Val Thr Val Thr Val Thr Glu Thr Glu Thr Glu Ser Val Ala Asp Gly | |
| 165 170 175 | |
| Gly Asp Lys Ser G1u Asn Asp Val Ala Leu Ser Pro Thr Ser Val Leu | |
| 180 185 190 | |
| Cys Asp Asn Asp Phe Ala Pro Phe Asp Asn Leu Gly Phe Cys Glu Val | |
| 195 200 205 | |
| Asp Ala Phe Gly Phe Asp Val Asp Ser Leu Phe Arg Leu Pro Asp Phe | |
| 210 215 220 | |
| Ala Met Thr Glu Lys Tyr Tyr Gly Asp Glu Phe Gly Glu Phe Asp Phe | |
| 225 230 235 240 | |
| Asp Asp Phe Ala Leu Glu Ala Arg | |
| 245 | |

The terms “protein” and “amino acid sequence” are used interchangeably herein to designate a plurality of amino acids linked in a serial array. Skilled artisans will recognize that through the process of mutation and/or evolution, proteins of different lengths and having differing constituents, e.g., with amino acid insertions, substitutions, deletions, and the like, may arise that are related to the proteins of the present invention by virtue of (a) amino acid sequence homology, and (b) good functionality with respect to pathogen resistance. Many deletions, insertions, and, especially, substitutions, are not expected to produce radical changes in the characteristics of the protein. However, when it is difficult to predict the exact effect of the substitution, deletion, or insertion in advance of doing so, one skilled in the art will appreciate that the effect may be evaluated by routine screening assays.

In addition to the above explicitly named proteins, therefore, the present invention also contemplates proteins having substantial identity to those set forth herein. The term “substantial identity,” as used herein with respect to an amino acid sequence, is intended to mean sufficiently similar to cause improved pathogen resistance when expressed in a plant transformed in accordance with the invention. In one preferred aspect of the present invention, variants having such potential modifications as those mentioned above, which have at least about 50% identity to the amino acid sequences set forth in SEQ ID NOS: 1, 2 and 3, are considered to have “substantial identitv” thereto. Sequences having lesser degrees of identity but comparable biological activity are considered to be equivalents. It is believed that the identity required to maintain proper functionality is related to maintenance of the tertiary structure of the protein such that specific interactive sequences will be properly located and will have the desired activity. As such, it is believed that there are discreet domains and motifs within the amino acid sequence which must be present for the protein to retain it advantageous functionality and specificity. While it is not intended that the present invention be limited by any theory by which it achieves its advantageous result, it is contemplated that a protein including these discreet domains and motifs in proper spatial context will retain good activity with respect to interaction with R gene products, even where substantial substitutions, insertions and/or deletions have taken place elsewhere in the sequence.

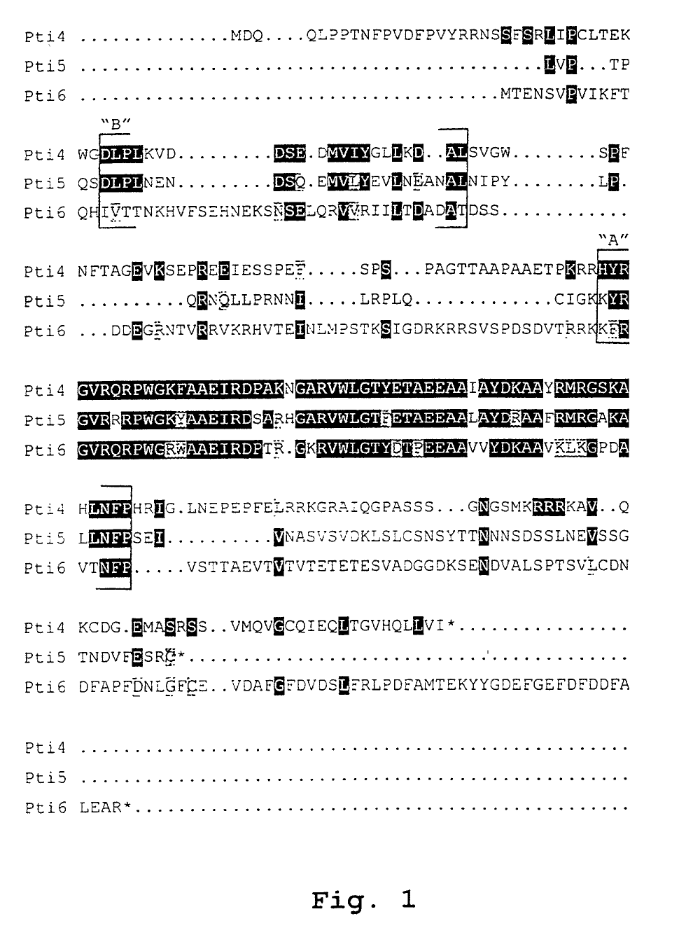

In this regard, a protein will find advantageous use according to the invention if it includes one or more amino acid consensus motifs and possesses substantially similar activity with respect to a protein set forth in SEQ ID NO:1, 2 or 3. The term “amino acid consensus motif” as used herein is intended to designate all or a portion of an inventive amino acid sequence which is substantially conserved among inventive proteins. For example, referring to FIG. 1, the box labeled “A” includes amino acid consensus 1 motif and includes generally the following sequence:

His/Lys Tyr/Phe Arg Gly Val Arg Gln/Arg Arg Pro
Trp Gly Lys/Arg Phe/Tyr/Trp Ala Ala Glu Ile Arg
Asp Pro/Ser Ala/Thr Lys/Arg --X-- Gly Ala/Lys Arg
Val Trp Leu Gly Thr Tyr/Phe Glu/Asp Thr Ala/Pro
Glu Glu Ala Ala --X-- Ala/Val Tyr Asp Lys/Arg
Ala Ala --X-- Arg/Lys Met/Leu Arg/Lys Gly
Ser/Ala Pro Lys/Asp Ala --X-- Leu/Thr Asn Phe Pro

wherein a “/” between two or in a series of amino acids indicates that any one of the amino acids indicated may be present at that location; and wherein “--X-- indicates that one or more amino acids may be present at that location, but not exceeding about 15 amino acids. The box labeled “B” includes amino acid consensus 2 motif and includes generally the following sequence:

Asp Leu Pro Leu --X-- Asp/Asn Ser Glu/Gln --X--
Met Val Ile/Leu/Val Tyr --X-- Leu --X-- Asp/Glu --
X-- Ala Leu

wherein a “/” between two or in a series of amino acids indicates that any one of the amino acids indicated may be present at that location; and wherein “--X--” indicates that one or more amino acids may be present at that location, but not exceeding about 15 amino acids. A protein comprising amino acid consensus 1 motif and/or amino acid consensus 2 motif and having substantially similar functionality to amino acid sequences set forth herein are intended to fall within the scope of the invention.

In a preferred aspect of the invention, nucleotide sequences encoding inventive proteins have the nucleotide sequences set forth below as SEQ ID NO:4 (Pti4), SEQ ID NO:5 (Pti5) and SEQ ID NO:6 (Pti6):

| | | |
|---|---|---|
| ATCACTAGAA AAAAAAACTA AAATTCAAAG CGA AAT GGA TCA ACA GTT ACC ACC | 54 | SEQ ID NO:4 |
| Met Asp Gln Gln Leu Pro Pro | | |
| 1 5 | | |
| GAC GAA CTT CCC GGT AGA TTT TCC GGT GTA TCG CCG GAA TTC AAG CTT | 102 | |
| Thr Asn Phe Pro Val Asp Phe Pro Val Tyr Arg Arg Asn Ser Ser Phe | | |
| 10 15 20 | | |
| CAG TCG TCT AAT TCC CTG TTT AAC TGA AAA ATG GGG AGA TTT ACC ACT | 150 | |
| Ser Arg Leu Ile Pro Cys Leu Thr Glu Lys Trp Gly Asp Leu Pro Leu | | |
| 25 30 35 | | |
| AAA AGT CGA CGA TTC CGA AGA TAT GGT AAT TTA CGG TCT ATT AAA AGA | 198 | |
| Lys Val Asp Asp Ser Glu Asp Met Val Ile Tyr Gly Leu Leu Lys Asp | | |
| 40 45 50 55 | | |
| CGC TCT AAG CGT CGG ATG GTC GCC GTT TAA TTT CAC CGC CGG CGA AGT | 246 | |
| Ala Leu Ser Val Gly Trp Ser Pro Phe Asn Phe Thr Ala Gly Glu Val | | |
| 60 65 70 | | |
| AAA ATC GGA GCC GAG AGA AGA AAT TGA ATC GTC GCC TGA ATT TTC ACC | 294 | |
| Lys Ser Glu Pro Arg Glu Glu Ile Glu Ser Ser Pro Glu Phe Ser Pro | | |
| 75 80 85 | | |
| TTC TCC GGC GGG AAC CAC GGC AGC TCC GGC GGC TGA AAC ACC GAA AAG | 342 | |
| Ser Pro Ala Gly Thr Thr Ala Ala Pro Ala Ala Glu Thr Pro Lys Arg | | |
| 90 95 100 | | |
| AAG ACA TTA TAG AGG CGT TAG ACA GCG TCC GTG GGG GAA ATT TGC GGC | 390 | |
| Arg His Tyr Arg Gly Val Arg Gln Arg Pro Trp Gly Lys Phe Ala Ala | | |
| 105 110 115 | | |
| GGA GAT TAG AGA TCC GGC GAA GAA CGG AGC TAG GGT TTG GCT TGG AAC | 438 | |
| Glu Ile Arg Asp Pro Ala Lys Asn Gly Ala Arg Val Trp Leu Gly Thr | | |
| 120 125 130 135 | | |
| GTA CGA AAC AGC TGA AGA AGC TGC AAT TGC TTA TGA TAA AGC TGC TTA | 486 | |
| Tyr Glu Thr Ala Glu Glu Ala Ala Ile Ala Tyr Asp Lys Ala Ala Tyr | | |
| 140 145 150 | | |
| TAG AAT GAG AGG ATC AAA AGC ACA TTT GAA TTT CCC GCA CCG GAT CGG | 534 | |
| Arg Met Arg Gly Ser Lys Ala His Leu Asn Phe Pro His Arg Ile Gly | | |
| 155 160 165 | | |
| TTT GAA TGA ACC GGA ACC GTT CGA GTT ACG GCG AAA AGG TCG AGC CAT | 582 | |
| Leu Asn Glu Pro Glu Pro Phe Glu Leu Arg Arg Lys Gly Arg Ala Ile | | |
| 170 175 180 | | |
| CCA AGG ACC GGC AAG CTC GTC GGG AAA CGG TTC CAT GAA ACG GAG AAG | 630 | |
| Gln Gly Pro Ala Ser Ser Ser Gly Asn Gly Ser Met Lys Arg Arg Arg | | |
| 185 190 195 | | |
| AAA AGC CGT TCA GAA ATG TGA TGG AGA AAT GGC GAG TAG ATC AAG TGT | 678 | |
| Lys Ala VaL Gln Lys Cys Asp Gly Glu Met Ala Ser Arg Ser Ser Val | | |
| 200 205 2l0 215 | | |
| CAT GCA AGT TGG ATG TCA AAT TGA ACA ATT GAC AGG TGT CCA TCA ACT | 726 | |
| Met Gln Val Gly Cys Gln Ile Glu Gln Leu Thr Gly Val His Gln Leu | | |
| 220 225 230 | | |
| ATT GGT CAT TTAAAAGCCG AATATTTCTC CGAACGCAAA ATACTATATT | 775 | |
| Leu Val Ile | | |
| ATTTTTCCAA ATTTATTGTA AATACGTAAT ACTCTATGAT AACGGAGAAA ATAGAAAGTT | 835 | |
| GAATTGGAAA AATATTGTGA TAGGGTTAAT CCAAAGTTGT AAAAAGTTTC ATTTTCATTA | 895 | |
| ATATTAATTT ACGTAAAAAA AAAAAAAAAA AAAAAAAA 933 | | |
| TCT GGT TCC AAC TCC TCA AAG TGA TTT ACC TCT TAA TGA GAA TGA CTC | 48 | SEQ ID NO:5 |
| Leu Val Pro Thr Pro Gln Ser Asp Leu Pro Leu Asn Glu Asn Asp Ser | | |
| 5 10 15 | | |
| ACA AGA GAT GGT ATT ATA TGA AGT TCT TAA TGA AGC TAA TGC TCT AAA | 96 | |
| Gln Glu Met Val Leu Tyr Glu Val Leu Asn Glu Ala Asn Ala Leu Asn | | |
| 20 25 30 | | |
| TAT TCC TTA TTT ACC CCA ACG AAA TCA ATT ACT CCC TAG AAA TAA TAT | 144 | |
| Ile Pro Tyr Leu Pro Gln Arg Asn Gln Leu Leu Pro Arg Asn Asn Ile | | |
| 35 40 45 | | |
| TCT TCG TCC ATT ACA GTG CAT AGG CAA GAA ATA CAG AGG AGT ACG ACG | 192 | |
| Leu Arg Pro Leu Gln Cys Ile Gly Lys Lys Tyr Arg Gly Val Arg Arg | | |
| 50 55 60 | | |
| TCG TCC GTG GGG GAA ATA CGC TGC GGA AAT TCG CGA TTC GGC TAG ACA | 240 | |
| Arg Pro Trp Gly Lys Tyr Ala Ala Glu Ile Arg Asp Ser Ala Arg His | | |
| 65 70 75 80 | | |
| TGG TGC GAG AGT ATG GCT AGG TAC GTT CGA AAC TGC TGA AGA AGC TGC | 288 | |
| Gly Ala Arg Val Trp Leu Gly Thr Phe Glu Thr Ala Glu Glu Ala Ala | | |
| 85 90 95 | | |
| GTT AGC TTA TGA TAG AGC GGC TTT TAG AAT GCG AGG TGC TAA GGC ACT | 336 | |
| Leu Ala Tyr Asp Arg Ala Ala Phe Arg Met Arg Gly Ala Lys Ala Leu | | |
| 100 105 110 | | |
| ACT TAA TTT TCC ATC TGA AAT AGT GAA CGC CTC TGT TTC AGT AGA CAA | 384 | |
| Leu Asn Phe Pro Ser Glu Ile Val Asn Ala Ser Val Ser Val Asp Lys | | |
| 115 120 125 | | |
| ATT AAG TTT GTG CTC AAA TAG TTA CAC TAC GAA TAA TAA TTC AGA TTC | 432 | |
| Leu Ser Leu Cys Ser Asn Ser Tyr Thr Thr Asn Asn Asn Ser Asp Ser | | |
| 130 135 140 | | |
| AAG TTT AAA TGA AGT TTC AAG TGG AAC TAA TGA TGT ATT TGA ATC AAG | 480 | |
| Ser Leu Asn Glu Val Ser Ser Gly Thr Asn Asp Val Phe Glu Ser Arg | | |
| 145 150 155 160 | | |
| ATG TTAAAACAGA GCTGTGCATG GAGAATTTCT TGGCACTCTA AGCGAATAAT | 533 | |
| Cys | | |
| GTGTGGACAC GTAGAAAATA TTTCTATTTA TGTAAGAATC AACTGAACTA TTAAAATTTC | 593 | |
| GTTGTTGTAT TTATATTATG TGCTTGCCTC TTCTCTTATT TTCCTTATGG AATTGTTTGC | 653 | |
| AGCGACGCAC GCTATAATCT CATGTAAAAA GATTGCTTAG GATACTTTAG TAGTATGTTT | 713 | |
| ATAAGTTGTA ATATACACCT TCTATTTTCT AAAAAAAAAA AAAAAAAA 761 | | |
| TTTGGCTTTA TACCTCTAAT TATATTGTTC TAATTATATG GTAGAAAGAT CTACTTCCCG | 60 | SEQ ID NO:6 |
| CCAAAAACAA CAAAGAAAGT AATCTCTTTT TCTTTGTTCA CTCATCAACT TGTTTCTCAA | 120 | |
| ATCATTTGTA TCACTGCAAC TTTTTCCACA CTTAAAAACT TTTTATACAA TAATATTGGT | 180 | |
| CACTATTCAC TCACTTCAAC CAGTTCTTGA TTGTTTTAGT ACTCCTTTTT GAGCTTATGA | 240 | |
| TGATTTTTTT TTGTGCTCTT TGAAAAAAAT ATCTTTTAAA TCGAACTGTA ACTTTAAGTT | 300 | |
| TTTGGTATAC 310 | | |
| CAT GAC GGA AAA TTC AGT TCC GGT GAT TAA ATT CAC TCA ACA CAT AGT | 358 | |
| Met Thr Glu Asn Ser Val Pro Val Ile Lys Phe Thr Gln His Ile Val | | |
| 5 10 15 | | |
| AAC TAC AAA CAA GCA TGT TTT TTC TGA GCA TAA CGA AAA ATC CAA TTC | 406 | |
| Thr Thr Asn Lys His Val Phe Ser Glu His Asn Glu Lys Ser Asn Ser | | |
| 20 25 30 | | |
| AGA GTT ACA AAG AGT TGT GAG GAT TAT ACT TAC AGA TGC CGA TGC TAC | 454 | |
| Glu Leu Gln Arg Val Val Arg Ile Ile Leu Thr Asp Ala Asp Ala Thr | | |
| 35 40 45 | | |
| AGA TTC TTC CGA TGA TGA AGG CCG GAA TAC TGT ACG GAG AGT GAA GAG | 502 | |
| Asp Ser Ser Asp Asp Glu Gly Arg Asn Thr Val Arg Arg Val Lys Arg | | |
| 50 55 60 | | |
| GCA CGT GAC GGA GAT CAA CCT TAT GCC GTC AAC CAA ATC GAT CGG CGA | 550 | |
| His Val Thr Glu Ile Asn Leu Met Pro Ser Thr Lys Ser Ile Gly Asp | | |
| 65 70 75 80 | | |
| CAG AAA ACG AAG ATC GGT GTC TCC GGA TTC TGA CGT CAC TCG TCG GAA | 598 | |
| Arg Lys Arg Arg Ser Val Ser Pro Asp Ser Asp Val Thr Arg Arg Lys | | |
| 85 90 95 | | |
| AAA GTT TAG AGG CGT TCG TCA AAG ACC GTG GGG TCG TTG GGC TGC AGA | 646 | |
| Lys Phe Arg Gly Val Arg Gln Arg Pro Trp Gly Arg Trp Ala Ala Glu | | |
| 100 105 110 | | |
| GAT TCG GGA CCC GAC CCG GGG AAA ACG GGT GTG GTT GGG TAC TTA TGA | 694 | |
| Ile Arg Asp Pro Thr Arg Gly Lys Arg Val Trp Leu Gly Thr Tyr Asp | | |
| 115 120 125 | | |
| CAC CCC AGA AGA AGC AGC TGT CGT TTA CGA TAA AGC TGC AGT TAA GCT | 742 | |
| Thr Pro Glu Glu Ala Ala Val Val Tyr Asp Lys Ala Ala Val Lys Leu | | |
| 130 135 140 | | |
| CAA AGG TCC TGA CGC CGT TAC CAA TTT TCC GGT ATC AAC AAC GGC GGA | 790 | |
| Lys Gly Pro Asp Ala Val Thr Asn Phe Pro Val Ser Thr Thr Ala Glu | | |
| 145 150 155 160 | | |
| GGT AAC GGT GAC GGT TAC GGA AAC CGA AAC CGA GTC TGT TGC CGA CGG | 838 | |
| Val Thr Val Thr Val Thr Glu Thr Glu Thr Glu Ser Val Ala Asp Gly | | |
| 165 170 175 | | |
| TGG AGA TAA AAG CGA AAA CGA TGT CGC TTT GTC ACC CAC CTC AGT TCT | 886 | |
| Gly Asp Lys Ser Glu Asn Asp Val Ala Leu Ser Pro Thr Ser Val Leu | | |
| 180 185 190 | | |
| CTG TGA CAA TGA TTT TGC GCC GTT TGA CAA TCT AGG GTT CTG CGA AGT | 934 | |
| Cys Asp Asn Asp Phe Ala Pro Phe Asp Asn Leu Gly Phe Cys Glu Val | | |
| 195 200 205 | | |
| GGA TGC TTT TGG TTT CGA CGT TGA TTC ACT TTT CCG GCT GCC GGA TTT | 982 | |
| Asp Ala Phe Gly Phe Asp Val Asp Ser Leu Phe Arg Leu Pro Asp Phe | | |
| 210 215 220 | | |
| TGC TAT GAC GGA GAA ATA CTA CGG CGA TGA ATT CGG CGA ATT TGA CTT | 1030 | |
| Ala Met Thr Glu Lys Tyr Tyr Gly Asp Glu Phe Gly Glu Phe Asp Phe | | |
| 225 230 235 240 | | |
| TGA CGA TTT TGC CCT TGA AGC TCG 1054 | | |
| Asp Asp Phe Ala Leu Glu Ala Arg | | |
| 245 | | |
| ATAGTGTACG AGGGGCTATT TCGTCCATTT TTGCAAATGG GTTCACTGGT TAGTTGACTA | 1114 | |
| GTGACGTGGC ATTTTTGGCG GGAATATATA TATAGTGATT AGCAGTCTCT ATTCATACGA | 1174 | |
| AGACTTTGTG AGAGATTTTT GTTTTTATTT TTCTGTTAAT TGTGGGTGAA TATTGTAATA | 1234 | |
| TGAAAAATTT TGTATGGTGA AATTGAATTA ATTAACGATG AAGATAAGGA GAGTGAAGGG | 1294 | |
| GGATGTGTGT ATTTTATGAT TGAGGTGTGT TTTTGTGATT CTGAAAAAAT AATTTATTAT | 1354 | |
| TTTACGTTGG AAATATAAAG TCAAAATTCT ATTGAAAAAA AAAAAAAAAA A 1405 | | |

the term “nucleotide sequence” is intended to refer to a natural or synthetic linear and sequential array of nucleotides and/or nucleosides, and derivatives thereof. Nucleotide sequences selected for use in accordance with the invention may be cloned from cDNA libraries corresponding to a wide variety of plant species. The present invention also contemplates nucleotide sequences having substantial identity to those set forth in SEQ ID NOS. 1, 2 and 3. The term “substantial identity” is used herein with respect to a nucleotide sequence to designate that the nucleotide sequence has a sequence sufficiently similar to one of those explicitly set forth above that it will hybridize therewith under moderately stringent conditions, this method of determining identity being well known in the art to which the invention pertains. Briefly, moderately stringent conditions are defined in Sambrook et al., Molecular Cloning: a Laboratory Manual, 2ed. Vol. 1, pp. 101-104, Cold Spring Harbor Laboratory Press (1989) as including the use of a prewashing solution of 5×SSC, 0.5% SDS, 1.0 mM EDTA (pH 8.0) and hybridization and washing conditions of about 55° C., 5×SSC. A further requirement of the term “substantial identity” as it relates to an inventive nucleotide sequence is that it must encode an inventive protein, i.e. one which is capable of physically interacting with an R gene product in a manner which enhances a plant's ability to resist pathogens.

Suitable DNA sequences according to the invention may be obtained, for example, by cloning techniques, these techniques being well known in the relevant art, or may be made by chemical synthesis techniques which are also well known in the art. Suitable nucleotide sequences may be isolated from DNA libraries obtained from a wide variety of species by means of nucleic acid hybridization or

PCR, using as hybridization probes or primers nucleotide sequences selected in accordance with the invention, such as those set forth in SEQ ID NOS: 4, 5 and 6; nucleotide sequences having substantial identity thereto; or portions thereof. In certain preferred aspects of the invention, nucleotide sequences from a wide variety of plant species may be isolated and/or amplified which encode Pti4/5/6, or proteins having substantial identity thereto and having excellent activity with respect to interaction with R gene products native to that species or R gene products of other plant species. It is expected that nucleotide sequences specifically set forth herein or selected in accordance with the invention may be advantageously used in a wide variety of plant species, including but not limited to a species from which it is isolated.

In certain preferred aspects of the invention, a PCR primer is selected for use as described above based upon the presence therein of a nucleotide consensus motif. The term “nucleotide consensus motif” as used herein is intended to designate all or a portion of an inventive nucleotide sequence, which encodes an amino acid sequence having substantial identity to an amino acid consensus motif (described herein). For example, a suitable nucleotide consensus motif, designated “nucleotide consensus 1 motif,” is one which encodes an amino acid sequence within the scope of amino acid consensus 1 motif. Another is “nucleotide consensus 2 motif,” which is a nucleotide sequence which encodes an amino acid sequence within the scope of amino acid consensus 2 motif.

It is readily understood that other nucleotide sequences may be advantageously selected for use in PCR primers designed to identify/isolate/amplify analogs to Pti4/5/6 in a wide variety of plant species. For instance, variations in a nucleotide consensus motif which are silent (i.e., do not result in the substitution of a different amino acid in the encoded protein), may advantageously be included in a nucleotide sequence used as a PCR primer in accordance with the invention.

DNA sequences selected for use in accordance with the invention can be incorporated into the genomes of plant or bacterium cells using conventional recombinant DNA technology, thereby making transformed plants having an enhanced ability to resist pathogen attack. In this regard, the term “genome” as used herein is intended to refer to DNA which is present in the plant or microorganism and which is heritable by progeny during propagation of the plant or microorganism. As such, inventive transgenic plants may alternatively be produced by breeding a transgenic plant made according to the invention with a second plant or selfing an inventive transgenic plant to form an F1 or higher generation plant. Transformed plants and progeny thereof are all contemplated by the invention and are all intended to fall within the meaning of the term “transgenic plant.”

Generally, transformation of a plant involves inserting a DNA sequence into an expression vector in proper orientation and correct reading frame. The vector contains the necessary elements for the transcription of the inserted protein-encoding sequences. A large number of vector systems known in the art can be advantageously used in accordance with the invention, such as plasmids, bacteriophage viruses or other modified viruses. Suitable vectors include, but are not limited to the following viral vectors: lambda vector system λgt11, λgt10, Charon 4, and plasmid vectors such as pBI121, pBR322, pACYC177, pACYC184, pAR series, pKK223-3, pUC8, pUC9, pUC18, pUC19, pLG339, pRK290, pKC37, pKC101, pCDNAII, and other similar systems. The DNA sequences are closed into the vector using standard cloning procedures in the art, as described by Maniatis et al., Molecular Cloning: A Laboratory Manual, Cold Springs Laboratory, Cold Springs Harbor, N.Y. (1982), which is hereby incorporated by reference. The plasmid pBI121 is available from Clontech Laboratories, Palo Alto, Calif. It is understood that related techniques may be advantageously used according to the invention to transform microorganisms such as, for example, Agrobacterium, yeast, E.coli and Pseudomonas.

In order to obtain efficient expression of the gene or gene fragment of the present invention, a promoter must be present in the expression vector. An expression vector according to the invention may be either naturally or artificially produced from parts derived from heterologous sources, which parts may be naturally occurring or chemically synthesized, and wherein the parts have been joined by ligation or other means known in the art. The introduced coding sequence is under control of the promoter and thus will be generally downstream from the promoter. Stated alternatively, the promoter sequence will be generally upstream (i.e. at the 5′ end) of the coding sequence. As such, in one representative example, enhanced Pti4/5/6 production may be achieved by inserting a Pti4/5/6 nucleotide sequence in a vector downstream from and operably linked to a promoter sequence capable of driving constitutive high-level expression in a host cell. Two DNA sequences (such as a promoter region sequence and a Pti-encoding sequence) are said to be operably linked if the nature of the linkage between the two DNA sequences does not (1) result in the introduction of a frame-shift mutation, (2) interfere with the ability of the promoter region sequence to direct the transcription of the desired Pti-encoding gene sequence, or (3) interfere with the ability of the desired Pti sequence to be transcribed by the promoter region sequence.

RNA polymerase normally binds to the promoter and initiates transcription of a DNA sequence or a group of linked DNA sequences and regulatory elements (operon). Promoters vary in their strength, i.e. their ability to promote transcription. Depending upon the host cell system utilized, a wide variety of suitable promoters can be used, and many are well known in the art. For example, a gene product may be obtained using a constitutive (e.g. Cauliflower Mosaic Virus 35S promoter), inducible (e.g. tomato E8 ethylene inducible promoter), developmentally regulated (e.g. Tomato polygalacturonase promoter) or tissue specific promoter to construct the vectors. Alternative promoters which may be suitably used in accordance with the invention include Figwort mosaic virus (FMV) promoter, Octopine synthase (OCS) promoter and also the native Pti4/5/6 promoter. It is not intended, however, that this list be limiting, but only provide examples of promoters which may be advantageously used in accordance with the present invention.

As briefly mentioned above, it is well known that there may or may not be other regulatory elements (e.g. enhancer sequences) which cooperate with the promoter and a transcriptional start site to achieve transcription of the introduced (i.e., foreign) sequence. The phrase “under control of” contemplates the presence of such other elements as are necessary to achieve transcription of the introduced sequence. Also, the recombinant DNA will preferably include a termination sequence downstream from the introduced sequence.

Once the defense gene of the present invention has been cloned into an expression system, it is ready to be transformed into a host cell, such as, for example, a plant cell. Plant tissue suitable for transformation in accordance with certain preferred aspects of the invention include whole plants, leaf tissues, flower buds, root tissues, meristems, protoplasts, hypocotyls and cotyledons. It is also understood, however, that this list is not intended to be limiting, but only provide examples of tissues which may be advantageously transformed in accordance with the present invention.

One technique of transforming plants with the gene conferring disease resistance in accordance with the present invention is by contacting the tissue of such plants with an inoculum of a bacteria transformed with a vector comprising a DNA sequence selected in accordance with the present invention. Generally, this procedure involves inoculating the plant tissue with a suspension of bacteria and incubating the tissue for about 48 to about 72 hours on regeneration medium without antibiotics at about 25-28° C.

Bacteria from the genus Agrobacterium may be advantageously utilized to transform plant cells. Suitable species of such bacterium include Agrobacterium tumefaciens and Agrobacterium rhizogenes, Agrobacterium tumefaciens (e.g., strains LBA4404 or EHA105) is particularly useful due to its well-known ability to transform plants. Another technique which may advantageously be used is vacuum-infiltration of flower buds using Agrobacterium-based vectors.

Another approach to transforming plant cells with a DNA sequence selected in accordance with the present invention involves propelling inert or biologically active particles at plant tissues or cells. This technique is disclosed in U.S. Pat. Nos. 4,945,050, 5,036,006 and 5,100,792, all to Sanford et al., which are hereby incorporated by reference. Generally, this procedure involves propelling inert or biologically active particles at the cells under conditions effective to penetrate the outer surface of the cell and to be incorporated within the interior thereof. When inert particles are utilized, the vector can be introduced into the cell by coating the particles with the vector. Alternatively, the target cell can be surrounded by the vector so that the vector is carried into the cell by the wake of the particle. Biologically active particles (e.g., dried yeast cells, dried bacterium or a bacteriophage, each containing DNA material sought to be introduced) can also be propelled into plant cells. It is not intended, however, that the present invention be limited by the choice of vector or host cell. It should of course be understood that not all vectors and expression control sequences will function equally well to express the DNA sequences of this invention. Neither will all hosts function equally well with the same expression system. However, one of skill in the art may make a selection among vectors, expression control sequences, and hosts without undue experimentation and without departing from the scope of this invention.

Once the recombinant DNA is introduced into the plant tissue, successful transformants can be screened using standard techniques such as the use of marker genes, e.g., genes encoding resistance to antibiotics. Additionally, the level of expression of the foreign DNA may be measured at the transcriptional level or as protein synthesized.

An isolated DNA sequence selected in accordance with the present invention may be utilized in an expression system to improve disease resistance in a wide variety of plant cells, including gymnosperms, monocots and dicots. These DNA sequences are particularly useful in crop plant cells such as rice, wheat, barley, rye, corn, potato, carrot, sweet potato, bean, pea, chicory, lettuce, cabbage, cauliflower, broccoli, turnip, radish, spinach, asparagus, onion, garlic, eggplant, pepper, celery, squash, pumpkin, zucchini, cucumber, apple, pear, quince, melon, plum, cherry, peach, nectarine, apricot, strawberry, grape, raspberry, blackberry, pineapple, avocado, papaya, mango, banana, soybean, tobacco, tomato, sorghum and sugarcane. According to one preferred aspect of the invention, the target plant is a tomato plant or a potato plant. According to another preferred aspect of the invention, the target plant is a monocot such as, for example, rice, wheat or corn. The present invention may also be used in conjunction with non-crop plants, such as, for example, Arabidopsis thaliana.

Those skilled in the art will recognize the agricultural advantages inherent in plants constructed to have increased or selectively increased expression of Pti4/516 and/or of nucleotide sequences which encode proteins having substantial identity thereto. Such plants are expected to have substantially improved resistance to pathogens and, therefore, will also be expected to have improved yield as compared to a corresponding non-transformed plant. Additionally, the present invention not only provides plants capable of minimizing immediate damage caused by pathogens, but is also useful to prevent the establishment of a strong pathogen population in a given area such as, for example, a given corn field.

The invention will be further described with reference to the following specific Examples. It will be understood that these Examples are illustrative and not restrictive in nature.

### EXAMPLE ONE

### Yeast Two-Hybrid Interaction of Pto with Pti4/5/6

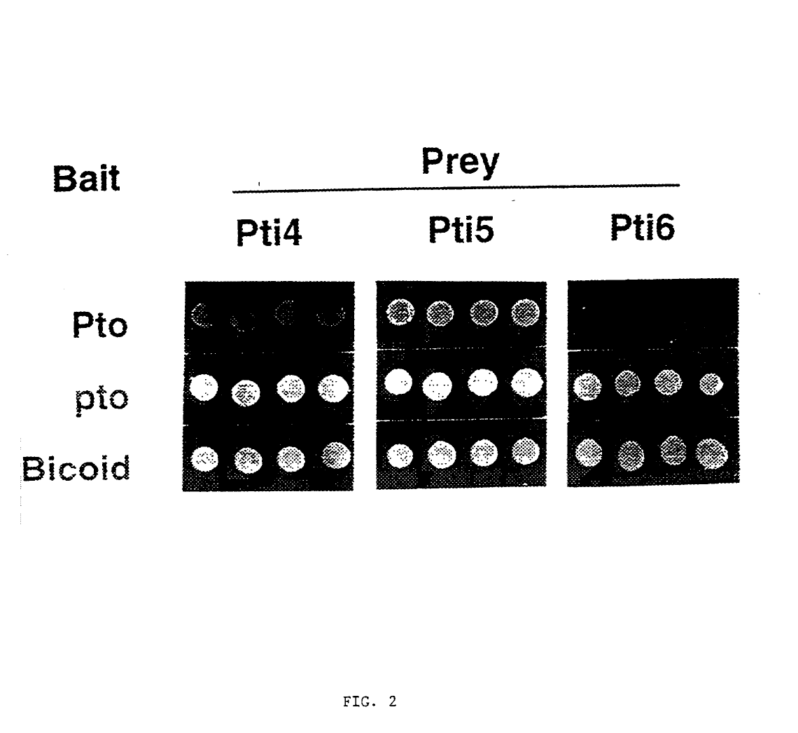

Yeast strains carrying the Pto bait and a prey of Pti4, Pti5 or Pti6 grew in the absence of leucine, indicative of the LEU2 reporter gene activation. When grown on X-Gal plates, these yeast cells were blue as a result of the lacZ reporter gene activation. As determined by the intensity of blue color, the strength of interaction of Pto with these three preys is in the order of Pti6>Pti4>Pti5. In contrast, control yeast strains expressing the arbitrary bait Bicoid and any one of the three preys did not activate the LEU2 or the LacZ reporter genes. FIG. 2 shows the specific interaction of Pti4, Pti5 and Pti6 with Pto in yeast. This test indicates that the interactions of these Pti proteins with Pto were specific.

### EXAMPLE TWO

### DNA Blot Analysis of Tomato Genomic DNA

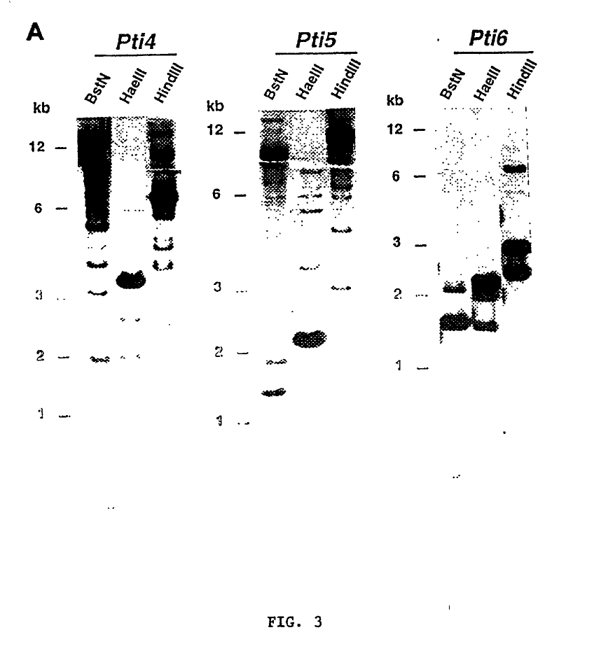

Genomic DNA (5 μg/lane) from Rio Grande-PtoR plants was digested with the indicated restriction enzymes, and the DNA blot was hybridized to the Pti456 cDNA probes. Results are set forth in FIG. 3 herein and deduced sequences are set forth herein as SEQ ID NOS: 4, 5 and 6

### EXAMPLE THREE

### Cloning of Pti4/5/6 Inserts into Fusion Protein Expression Vectors in E. coli

The Ptil cDNA was removed from the GST-Ptil fusion plasmid (Zhou, J., Loh, Y. -T., Bressan, R. A. and Martin, G. (1995). The tomato gene Ptil encodes a serine/threonine kinase that is phosphorylated by Pto and is involved in the hypersensitive response. Cell 83, 925-935.) with EcoRI and XhoI and replaced with cDNA inserts of Pti4/5/6 to create GST-Pti4/5/6 fusion constructs. Pti4 cDNAs (nucleotides 13-993) and Pti5 cDNA (nucleotides 82-782) were excised form pJG4-5 with EcoRI and XhoI before ligation into the pGEX vector. The full length Pti6 insert was PCR-amplified using the full length Pti6 cDNA clone in pBluescript SK (−) (Stratagene) as a template and the upstream primer 5′-GAGAATTCATGACGGAAA ATTCAG-3′ and the T7 primer 5′-AATACGACTCACTATAG-3′. The PCR product was first digested partially with EcoRI and then digested completely with XhoI before being inserted into the GST-expression vector. The resulting constructs were introduced into E. coli strain PR745 (Ion-New England Biolabs, Beverly, Mass.), and GST-fusion proteins were expressed and purified as described by Guan, K. -L., and Dixon, J. E. (1991). Eukaryotic proteins expressed in Escherichia coli: an improved thrombin cleavage and purification of fusion proteins with glutathione S-transferase, Anal. Biochem. 192, 262-267.

### EXAMPLE FOUR

### Gel-Mobility Shift Assay

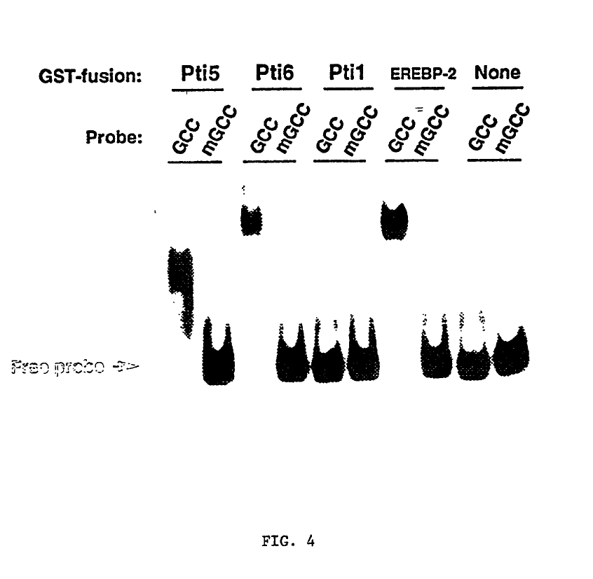

The wild type gln2 PR-box 2x (CATAAGAGCCGCCACTAAAATAA to GACCGATCAAATAAGAGCCGCCAT) and mutated PR-box 2x (CATAAGATCCTCCACTAAAATAAGACCGATCAAATAAGATCCTCCAT) were end-labeled by 32P as described by Ausubel, F. M., Brent, R., Kingston, R. E., Moore, D. D., Seidman, J. G., Smith, J. A., and Struhl, K. (1994). Current Protocols in Molecular Biology. (New York: Greene Publish Associates/John Wiley and Sons). Four fmol of probe was mixed with each of the purified GST-fusion proteins in a buffer containing 2 μg poly(dA-dT) (dA-dT), 25 mM Hepes (PH7.5), 40 mM KCl, 0.1 mM EDTA, 10% glycerol, and 1 mM DTT, incubated at room temperature for 15 minutes, and electrophoresed on a 4% polyacrylamide gel in 0.25×TBE buffer. Ohme-Takagi, M. and Shinshi, H. (1995). Ethylene-inducible DNA-binding proteins that interact with an ethylene-responsive element. Plant Cell 7, 173-182. The gel was subsequently dried and exposed to x-ray film. As shown in FIG. 4, both GST-Pti5 and GST-Pti6 bound the wild type PR-box. No binding was detected when the mutated PR-box was used in the assay, indicating that binding of GST-Pti5 and GST-Pti6 to the PR-box was highly specific. In contrast to GST-Pti5 and GST-Pti6, neither GST-Ptil nor GST itself bound to the PR-box. These results further confirmed the specificity of binding of Pti5 and Pti6 to the gln2 PR-box.

### EXAMPLE FIVE

### Plant Inoculation and RNA Blot Analysis

Leaves of 7-week old tobacco plants were injected with P.s. tabaci strain 11528R race 0 or the same strain carrying the avrPto gene in pPTE6 (Ronald, P. C., Salmeron, J. M., Carland, F. M., and Staskawicz, B. J. (1992). The cloned avirulence gene avrPto induces disease resistance in tomato cultivars containing the Pto resistance gene. J. Bacteriol. 174, 1604-1611.) at 10⁶ cfu/ml or 10⁸ cfu/ml, harvested at various time points following inoculation, and total RNA was extracted. Ten μg RNA per sample was separated on 1% formaldehyde agarose gel, and duplicate RNA blots were hybridized to the following probes as described by Zhou, J., Loh, Y. -T., Bressan, R. A. and Martin, G. (1995). The tomato gene Pti1 encodes a serine/threonine kinase that is phosphorylated by Pto and is involved in the hypersensitive response. Cell 83, 925-935.: PRP1, CHN50, and Osmotin.

What is claimed is:

1. An isolated DNA sequence comprising a nucleotide sequence having substantial identity to the nucleotide sequence of SEQ ID NO:4, SEQ ID NO:5 or SEQ ID NO:6.

2. An isolated protein comprising an amino acid sequence having substantial identity to the amino acid sequence of SEQ ID NO:1, SEQ ID NO:2 or SEQ ID NO:3.

3. A vector useful for transforming a cell, said vector comprising a nucleotide sequence having substantial identity to the nucleotide sequence of SEQ ID NO:4, SEQ ID NO:5 or SEQ ID NO:6; and regulatory elements flanking the nucleotide sequence, the regulatory elements being effective to control expression of the sequence in a cell.

4. A plant transformed with the vector of claim 3, or progeny thereof, the plant being capable of expressing the nucleotide sequence.

5. The plant according to claim 4, the plant being selected from the group consisting of monocots or dicots.

6. A microorganism transformed with the vector of claim 3, the microorganism being capable of expressing the nucleotide sequence.

7. The microorganism according to claim 6, wherein the microorganism is selected from the group consisting of Agrobacterium, yeast, E. coli and Pseudomonas.

8. A method for enhancing a plant's ability to resist pathogens, comprising:
providing a vector comprising a nucleotide sequence encoding a protein, and regulatory elements flanking the nucleotide sequence, the regulatory elements being effective to control expression of the nucleotide sequence in a target plant; and
transforming the target plant with the vector to provide a transformed plant;
wherein the protein comprises an amino acid sequence having substantial identity to amino acid concensus 1 motif; and
wherein the transformed plant is capable of expressing the nucleotide sequence.

9. The method according to claim 8, wherein the target plant is selected from the group consisting of monocots and dicots.

10. The method according to claim 8, wherein the nucleotide sequence has substantial identity to the nucleotide sequence of SEQ ID NO:4, SEQ ID NO:5 or SEQ ID NO:6.

11. The method according to claim 8, wherein the regulatory elements include a plant promoter.

12. A transgenic plant obtained according to the method of claim 8 or progeny thereof.

13. A method for transforming a target cell, comprising:
providing a DNA sequence vector comprising a nucleotide sequence having substantial identity to nucleotide consensus 1 motif, and regulatory elements flanking the nucleotide sequence, the regulatory elements being effective to allow expression of the nucleotide sequence in a target cell; and
transforming the target cell with the vector to provide a transformed cell, wherein the transformed cell is capable of expressing the nucleotide sequence.

14. The method according to claim 13, wherein the nucleotide sequence has substantial identity to the nucleotide sequence of SEQ ID NO:4, SEQ ID NO:5 or SEQ ID NO:6.

15. The method according to claim 13, wherein the target cell is a selected from the group consisting of a plant cell, an E. coli cell, a yeast cell, an Agrobacterium cell or a Pseudomonas cell.

16. A transgenic cell prepared according to the method of claim 13.

17. A method of producing a transformed plant, comprising incorporating into the nuclear genome of the plant an isolated nucleotide sequence which encodes protein comprising an amino acid sequence having substantial identity to amino acid consensus 1 motif to provide a transformed plant capable of expressing the protein in an amount effective to enhance the ability of the transformed plant to resist pathogens.

18. The method according to claim 17, wherein the protein further comprises an amino acid sequence having substantial identity to amino acid consensus 2 motif.

19. The method according to claim 17, wherein the protein has an amino acid sequence having substantial identity to the amino acid sequence of SEQ ID NO:1, SEQ ID NO:2 or SEQ ID NO:3.

20. An isolated protein comprising an amino acid sequence having substantial identity to amino acid consensus 1 motif, provided that said isolated protein is capable of interacting with proteins encoded by a resistance gene.

21. The isolated protein according to claim 20, wherein said isolated protein further comprises an amino acid sequence having substantial identity to amino acid consensus 2 motif.

22. A primer for amplifying a DNA sequence having substantial identity to Pti4, Pti5 or Pti6, comprising a nucleotide sequence having substantial identity to nucleotide consensus 1 motif.

23. A primer for amplifying a DNA sequence having substantial identity to Pti4, Pti5 or Pti6, comprising a nucleotide sequence having substantial identity to nucleotide consensus 2 motif.
